(19) United States
(12) Patent Application Publication
(10) Pub. No.: US 2012/0230924 A1
(43) Pub. Date: Sep. 13, 2012

(54) Indoline Derivatives And Their Use As Flavouring Agents

(76) Inventor: David C. Bom, Cincinnati, OH (US)

(21) Appl. No.: 13/503,127

(22) PCT Filed: Oct. 28, 2010
(86) PCT No.: PCT/EP2010/066379

§ 371 Date: May 15, 2012

Related U.S. Application Data
(60) Provisional application No. 61/256,513, filed on Oct. 30, 2009.

(51) Int. Cl. A23L 1/236; A21D 13/00; A61Q 11/00; A23L 2/56; C07D 209/08; A61K 8/49; A23G 3/36; A23C 9/156

(52) U.S. Cl. 424/54; 426/537; 426/442; 548/490

(57) ABSTRACT

Organic compounds of formula (I)

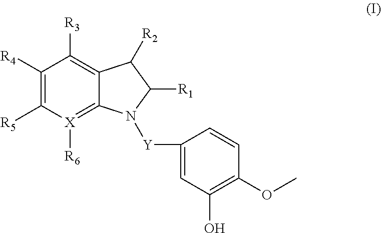

wherein:
X is N, or C,
R₁₋R₆ may be independently selected from the group consisting of, H, substituted, unsubstituted, branched or unbranched C₁-C₄ alkyl, methoxy, ethoxy, Br, F, Cl, provided that when X is nitrogen there is no substituent at R₆
Y is an alkylene group of the formula (CR₇R₈)ₘ wherein
R₇ and R₈ may be independently selected from the group consisting of hydrogen, substituted, unsubstituted, branched or unbranched C₁-C₄ alkylene, and m is an integer selected from 1 to 6.

The compounds create or modify flavour, in particular sweet flavour and may be used in compositions and consumable products.

This invention relates to compounds that create or modify flavour, to methods of their manufacture, and to compositions, and consumable products containing said compounds.

In the flavour industry there is a constant demand for new compounds that create or modify the flavour of compositions and consumable products.

Such compounds extend a flavourist's palette and result in greater product diversity for consumers.

In particular there is demand for compounds that are able to create or modify the sweet flavour of compositions and consumable products. Such compounds may replace, or reduce reliance on conventional sugars the use of which can be undesirable due to health and wellness concerns such as diabetes and obesity.

Furthermore, sweet-tasting materials may be useful for masking bitter tastes associated with certain compositions and consumable products, such as pharmaceutical compositions and products, leading to increased acceptance by users.

The present invention relates to compounds of formula (I), as defined hereinbelow, and to the use of said compounds to create, or modify the flavour, in particular the sweet flavour of compositions and consumable products.

The present invention also relates to methods of creating, enhancing or modifying the flavour, in particular the sweet flavour of compositions and consumable products by adding to said compositions or consumable products at least one compound of formula (I).

Furthermore the present invention relates to compositions and consumable products comprising said compounds of formula (I).

The term “consumable product” as used herein refers to composition that may be placed in the oral cavity and ingested or that may be placed in the oral cavity before being discarded, for example mouthwash and chewing gum.

The present invention can be understood more readily by reference to the following detailed description of the various embodiments, and to the examples included herein.

In a first aspect of the present invention there are provided compounds of formula (I).

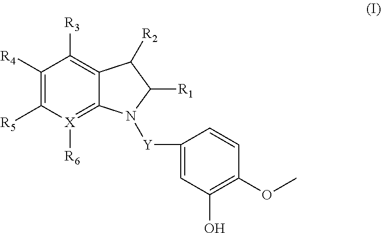

wherein:

X is N, or C,

R₁₋R₆ may be independently selected from the group consisting of, H, substituted, unsubstituted, branched or unbranched C₁-C₄ alkyl, methoxy, ethoxy, Br, F, Cl, provided that when X is nitrogen there is no substituent at R₆

Y is an alkylene group of the formula (CR₇R₈)ₘ wherein

R₇ and R₆ may be independently selected from the group consisting of hydrogen, substituted, unsubstituted, branched or unbranched C₁-C₄ alkylene, and m is an integer selected from 1 to 6.

In a particular embodiment X is C.

In a particular embodiment R₁₋R₆ are independently selected from the group consisting of H, methyl, methoxy, F.

In a particular embodiment Y is CH_{2.}

In a more particular embodiment still compounds of formula (I) may be selected from the group consisting of: 5-(indolin-1-ylmethyl)-2-methoxyphenol, 2-methoxy-5-((7-methylindolin-1-yl)methyl)phenol, 2-methoxy-5-((4-methylindolin-1-yl)methyl)phenol, 2-methoxy-5-((6-methylindolin-1-yl)methyl)phenol, 2-methoxy-5((5-methylinolin-1-yl)methyl)phenol.

Compounds of the formula (I) can be formed by known methods using commercially available starting materials, reagents and solvents.

Compounds of formula (I) may exist as a mixture of stereoisomers. Such stereoisomers of the compounds of formula (I) may be used as a racemic mixture, or the mixture may be resolved into its stereoisomers by techniques generally known in the art. The use of the term “a compound” of formula (I) may refer to both a racemic mixture and the individually isolated isomers.

Compounds of formula (I) may be prepared by reduction of the appropriately substituted indole derivative with isovanillin. Non limiting examples of common reducing agents include pyridine borane, sodium cyanoborohydride, triacetoxyborohydride and reagents commonly used for catalytic hydrogenation.

A more particular process for preparing compounds of the formula (I) is set out schematically below.

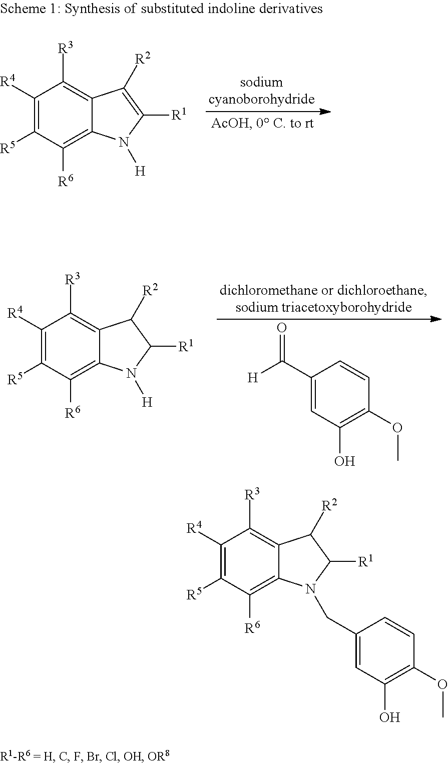

The process proceeds with the reduction of an appropriately substituted indole using sodium cyanoborohydride and acetic acid. The resulting indoline is then treated with isovanillin and sodium triacetoxyborohydride. The reaction is conducted in the presence of an inert solvent at room temperature. Suitable solvents for this reaction include, but are not limited to, dichloromethane and dichloroethane.

Further examples of suitable reaction conditions are provided in the examples.

The applicant has found that compounds of formula (I) may be used to create or modify the flavour, in particularly the sweet flavour, of compositions and consumable products.

In another aspect of the present invention there is provided a method of creating, or modifying the flavour of a composition comprising the step of adding to said composition, at least one compound of formula (I).

The compounds of formula (I) may be added into a composition in neat form, or in a solvent, or they may first be modified, for example by entrapped with an entrapment material such as for example polymers, capsules, microcapsules, nanocapsules, liposomes, precursors, film formers, absorbents such as for example by using carbon or zeolites, cyclic oligosaccharides and mixtures thereof, or they may be chemically bound to substrates which are adapted to release the compounds of formula (I) upon application of an exogenous stimulus such as light, enzymes, or the like.

A compound of formula (I) may be used as the sole flavouring component in a composition. Alternatively a compound of formula (I) may be employed in conjunction with other compounds of formula (I) and/or other flavourant ingredients known in the art, in particularly sweeteners, and sweetener enhancers.

Compounds of formula (I) may be used in a compositions at a concentration of up to 100% by weight of the flavour components of the composition. However, more commonly compounds of formula (I) will be used with other flavour ingredients, in particular sweeteners and sweetener enhancers, at a concentration of 0.01% to 99.9% by weight of the flavour components of the composition.

Sweeteners and sweetener enhances include, but are not limited to, fructose, glucose, sucrose, lactose, maltose, saccharin, aspartame, sucralose, neotame, sorbitol, xylitol, maltodextrol, polyols, neohesperidin dihydrochalcone, rebaudioside, stevioside, neotame, mannitol, erithrytol, xylose, rhamnose, Luo Han Guo extract, mogriside (V), stevia extract, thaumatin, inositol, and trilobatin.

Other flavourant ingredients that may be used with compounds of formula (I) include, but are not limited to, natural flavours, artificial flavours, spices, seasonings, and the like. Exemplary flavouring ingredients include synthetic flavour oils and flavouring aromatics and/or oils, oleoresins, essences, distillates, and extracts derived from plants, leaves, flowers, fruits, and so forth, and combinations comprising at least one of the foregoing.

Exemplary flavour oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavouring agents include artificial, natural and synthetic fruit flavours such as vanilla, and citrus oils including lemon, orange, lime, grapefruit, yazu, sudachi, and fruit essences including apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya and so forth. Additional exemplary flavours imparted by a flavouring agent include a milk flavour, a butter flavour, a cheese flavour, a cream flavour, and a yogurt flavour; a vanilla flavour; tea or coffee flavours, such as a green tea flavour, an oolong tea flavour, a tea flavour, a cocoa flavour, a chocolate flavour, and a coffee flavour; mint flavours, such as a peppermint flavour, a spearmint flavour, and a Japanese mint flavour; spicy flavours, such as an asafetida flavour, an ajowan flavour, an anise flavour, an angelica flavour, a fennel flavour, an allspice flavour, a cinnamon flavour, a chamomile flavour, a mustard flavour, a cardamom flavour, a caraway flavour, a cumin flavour, a clove flavour, a pepper flavour, a coriander flavour, a sassafras flavour, a savory flavour, a Zanthoxyli Fructus flavour, a perilla flavour, a juniper berry flavour, a ginger flavour, a star anise flavour, a horseradish flavour, a thyme flavour, a tarragon flavour, a dill flavour, a capsicum flavour, a nutmeg flavour, a basil flavour, a marjoram flavour, a rosemary flavour, a bayleaf flavour, and a wasabi (Japanese horseradish) flavour; a nut flavour such as an almond flavour, a hazelnut flavour, a macadamia nut flavour, a peanut flavour, a pecan flavour, a pistachio flavour, and a walnut flavour; alcoholic flavours, such as a wine flavour, a whisky flavour, a brandy flavour, a rum flavour, a gin flavour, and a liqueur flavour; floral flavours; and vegetable flavours, such as an onion flavour, a garlic flavour, a cabbage flavour, a carrot flavour, a celery flavour, mushroom flavour, and a tomato flavour.

In some embodiments, said other flavourant ingredients include aldehydes and esters such as cinnamyl acetate, cinnamaldehyde, citral diethylacetal, dihydrocarvyl acetate, eugenyl 49 formate, p-methylamisol, and so forth can be used. Further examples of aldehyde flavourings include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavours), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like.

Further examples of other flavourant ingredients can be found in “Chemicals Used in Food Processing”, publication 1274, pages 63-258, by the National Academy of Sciences.

Compounds of formula (I) can additionally be used in compositions, as described hereinabove, in conjunction with one or more ingredients or excipients conventionally used in compositions, for example carrier materials and other auxiliary agents commonly used in the art. Suitable excipients for compositions are well known in the art and include, for example, without limitation, solvents (including water, alcohol, ethanol, oils, fats, vegetable oil, and miglyol), binders, diluents, disintegranting agents, lubricants, flavouring agents, coloring agents, preservatives, antioxidants, emulsifiers, stabilisers, flavour-enhancers, anti-caking agents, and the like.

Examples of such carriers or diluents for compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

Other suitable and desirable ingredients of compositions are described in standard texts, such as “Handbook of Industrial Chemical Additives”, ed. M. and I. Ash, 2^{nd} Ed., (Synapse 2000).

In a further aspect of the present invention there is provided a method of creating, or modifying the flavour, in particularly the sweet flavour, of a consumable product comprising the the step of adding to said consumable product at least one compound of formula (I).

Compounds of formula (I), or compositions containing compounds of formula (I) can be added to consumable products by using conventional techniques to directly admix said compound or composition into the consumable product.

The quantities in which compounds of formula (I) may be added to consumable products may vary within wide limits and depend, inter alia, on the nature of the consumable product, on the effect desired, the purpose of adding compounds of formula (I) to a consumable product, for example masking a bitter taste, or enhancing a sweet taste, and on the nature and quantity of any other components of the consumable product. It is well within the purview of the person skilled in the art to decide on suitable quantities of compounds of formula (I) to incorporate into a consumable product depending on the end use and effect required.

Typical, non limiting, concentrations of compounds of formula (I), in ppm by weight based on the weight of the consumable product, are: 500 ppm to 0.01 ppm, more particularly 250 ppm to 0.01 ppm, still more particularly 100 ppm to 1 ppm.

The compounds of formula (I) can be added to all manner of consumable products. Examples include, but are not limited to, foodstuffs of all kinds, confectionery products, baked products, sweet products, savory products, dairy products, beverages and oral care products.

Exemplary foodstuffs include, but are not limited to, chilled snacks, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, dried food, dessert mixes, sauces, dressings and condiments, herbs and spices, spreads, jams and preserves, honey, chocolate spreads, nut-based spreads, and yeast-based spreads.

Exemplary confectionery products include, but are not limited to, chewing gum (which includes sugarized gum, sugar-free gum, functional gum and bubble gum), centerfill confections, chocolate and other chocolate confectionery, medicated confectionery, lozenges, tablets, pastilles, mints, standard mints, power mints, chewy candies, hard candies, boiled candies, breath and other oral care films or strips, candy canes, lollipops, gummies, jellies, fudge, caramel, hard and soft panned goods, toffee, taffy, liquorice, gelatin candies, gum drops, jelly beans, nougats, fondants, combinations of one or more of the above, and edible compositions incorporating one or more of the above.

Exemplary baked products include, but are not limited to, alfajores, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes,

Exemplary sweet products include, but are not limited to, breakfast cereals, ready-to-eat (“rte”) cereals, family breakfast cereals, flakes, muesli, other ready to eat cereals, children's breakfast cereals, hot cereals,

Exemplary savory products include, but are not limited to, salty snacks (potato chips, crisps, nuts, tortilla-tostada, pretzels, cheese snacks, corn snacks, potato-snacks, ready-to-eat popcorn, microwaveable popcorn, pork rinds, nuts, crackers, cracker snacks, breakfast cereals, meats, aspic, cured meats (ham, bacon), luncheon/breakfast meats (hotdogs, cold cuts, sausage), tomato products, margarine, peanut butter, soup (clear, canned, cream, instant, UHT), canned vegetables, pasta sauces.

Exemplary dairy products include, but are not limited to, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavoured, functional and other condensed milk, flavoured milk drinks, dairy only flavoured milk drinks, flavoured milk drinks with fruit juice, soy milk, sour milk drinks, fermented dairy drinks, coffee whiteners, powder milk, flavoured powder milk drinks, cream, yoghurt, plain/natural yoghurt, flavoured yoghurt, fruited yoghurt, probiotic yoghurt, drinking yoghurt, regular drinking yoghurt, probiotic drinking yoghurt, chilled and shelf-stable desserts, dairy-based desserts, soy-based desserts.

Exemplary beverages include, but are not limited to, flavoured water, soft drinks, fruit drinks, coffee-based drinks, tea-based drinks, juice-based drinks (includes fruit and vegetable), milk-based drinks, gel drinks, carbonated or non-carbonated drinks, powdered drinks, alcoholic or non-alcoholic drinks.

There now follows a series of non-limiting examples that serve to illustrate the invention.

### EXAMPLE 1

### 5-(indolin-1-ylmethyl)-2-methoxyphenol

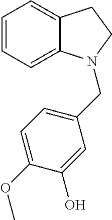

5 Indoline (1 g, 8 mmol) and isovanillin (1.28 g, 8.39 mmol) were dissolved in dichloroethane (42 ml) and cooled to 0° C. Next, sodium triacetoxyborohydride (1.78 g, 8.39 mmol) was added portion wise and, after 10 min, the reaction was warmed to rt. The reaction was monitored by TLC (50:50 hexane:EtOAc). After 5 h, the reaction was poured into ice cooled saturated NaHCO₃ (100 ml) and extracted with dichloromethane (100 ml). The organic layer was washed with brine (100 ml), dried (MgSO₄) and concentrated providing a brown oil. This oil was purified by flash chromatography on SiO₂ using a gradient of 100% hexane to 75% hexane:25% MTBE providing a light pink solid. This solid was suspended in a minimum amount of dichloromethane, sonicated and treated with hexane. This material was suction filtered, washed with hexane and dried in Vacuo providing the above titled product (1.7 g, 79.3% yield) as a white solid.

¹H NMR (CDCl₃; 300 MHz) δ 2.93 (t, J=8 Hz, 2H); 3.27 (t, J=8 Hz, 2H); 3.85 (s, 3H); 4.13 (s, 2H); 5.61 (s, 1H); 6.49 (d, J=8 Hz, 1H); 6.64 (ddd, J₁=8 Hz, J₂=1 Hz, 1H); 6.77-6.85 (m, 2H); 6.94 (d, J=2 Hz, 1H); 7.01-7.08 (m, 2H); ¹³C NMR (CDCl₃; 75 MHz) δ 28.5, 53.2, 53.5, 56.0, 107.1, 110.6, 114.3, 117.6, 119.4, 124.5, 127.3, 130.1, 131.8, 145.71, 145.74, 152.6; MS calculated for C16H17NO2+H 256, observed 256.

### EXAMPLE 2

### 5-((5-bromoindolin-1-yl)methyl)-2-methoxyphenol

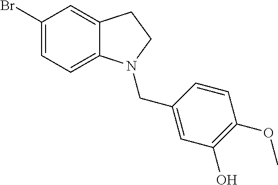

Following the same procedure as outlined in example 1, 5-bromoindoline (3 g, 15.2 mmol) and isovanillin (2.31 g, 15.2 mmol) were dissolved in dichloromethane (75 ml) and treated with sodium triacetoxyborohydride (3.22 g, 15.2 mmol). Work up and chromatography provided the above titled product (2.44 g, 81% yield) as a tan solid.

¹H NMR (CDCl₃; 300 MHz) δ 3.80 (s, 2H); 3.88 (s, 3H); 3.91 (s, 4H); 5.75 (br s, 1H); 6.78-6.83 (m, 1H); 6.89 (dd, J=₁=8 Hz, J₂=2 Hz, 1H); 6.98 (d, J=2 Hz, 1H); 7.16 (s, 4H); ¹³C NMR (CDCl₃; 75 MHz) δ 56.0, 58.9, 59.8, 110.6, 115.2, 120.2, 122.3. 126.6, 132.4, 140.3, 145.6, 145.8; MS calculated for Cl 6H 17NO2+H 256, observed 256.

### EXAMPLE 3

### 2-methoxy-5-((7-methylindolin-1-yl)methyl)phenol

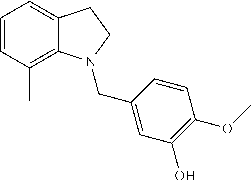

### a.) 7-methylindoline

Glacial acetic acid (100 ml) was added to a 500 ml round bottom flask (rbf) containing 7-methylindole (5 g, 38.2 mmol) and the contents were cooled to 0° C. Next, sodium cyanoborohydride (7.24 g, 115 mmol) was added portionwise and the contents were stirred for 15 min. at 0° C. and then warmed to rt and stirred for 3 h. Next, the reaction mixture was poured into ice water (400 ml) and carefully treated with 50% NaOH until the pH was 14. The reaction mixture was extracted with MTBE (2×300 ml) and the organic layer was washed with water (2×250 ml) followed by brine (1×250 ml). The organic layer was dried (K₂CO₃), filtered and concentrated. This product was used in the next reaction without further purification. Structure and purity was confirmed by GC/MS. The retention time was 4.38 min. The MS was calculated for C9H11N 133, observed 133.

### b.) 2-methoxy-5-((7-methylindolin-1-yl)methyl)phenol

Following the same procedure as outlined in example 1, 7-methylindoline (1 g, 7.52 mmol) and isovanillin (1.14 g, 7.52 mmol) were dissolved in dichloromethane (38 ml) and treated with sodium triacetoxyborohydride (1.59 g, 7.52 mmol). Work up and chromatography provided the above titled product (0.58 g, 29% yield) as a white solid.

¹H NMR (CDCl₃; 300 MHz) δ 2.34 (s, 3H), 2.92 (t. J=9 Hz, 2H), 3.31 (t, J=9 Hz, 2H), 3.86 (s, 3H), 4.33 (s, 2H), 5.64 (br s. 1H), 6.67 (t, J=7 Hz, 1H), 6.77-6.82 (m, 2H), 6.84-6.87 (m, 1H), 6.94-6.97 (m, 2H); ¹³C NMR (CDCl₃; 75 MHz) δ 19.5, 29.1, 53.9, 55.9, 56.2, 110.8, 114.2, 119.2, 119.5, 120.4, 122.6, 130.8, 131.5, 133.2, 145.8, 145.8, 150.9; MS calculated for C17H19NO2+H 270, observed 270.

### EXAMPLE 4

### 2-methoxy-5-((2-methylindolin-1-yl)methyl)phenol

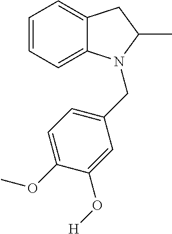

Following the same procedure as outlined in example 1, 2-methylindoline (2 g, 15 mmol) and isovanillin (2.28 g, 15 mmol) were dissolved in dichloromethane (75 ml) and treated with sodium triacetoxyborohydride (3.18 g, 15 mmol). Work up and chromatography provided the above titled product (2.04 g, 50% yield) as a tan oil.

¹H NMR (CDCl₃; 300 MHz) δ 1.28 (d, J=6 Hz, 3H), 2.64 (dd, J₁=15 Hz, J₂=9 Hz, 1H), 3.13 (dd, J₁=15 Hz, J₂=9 Hz, 1H), 3.66-3.74 (m, 1H), 3.85 (s, 3H), 4.07 (d, J=16 Hz, 1H), 4.27 (d, J=16 Hz, 1H), 5.59 (s, 1H), 6.32 (d, J=8 Hz, 1H), 6.60 (ddd, J₁=8 Hz, J₂=1 Hz, 1H), 6.75-6.86 (m, 2H), 6.92-7.03 (m, 3H); ¹³C NMR (CDCl₃; 75 MHz) δ 19.7, 37.5, 50.7, 56.2, 60.5, 107.0, 110.8, 113.9, 117.4, 118.9, 124.2, 127.5, 128.9, 132.7, 145.7, 145.8, 152.8; MS calculated for C17H19NO2+H 270, observed 270.

### EXAMPLE 5

### 2-methoxy-5-((3-methylindolin-1-yl)methyl)phenol

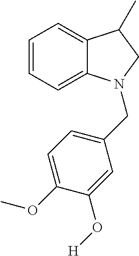

### a.) 3-methylindoline

Following the same procedure outlined in step a of example 3, 3-methylindole (5 g, 38.2 mmol) in glacial acetic acid (100 ml) was treated with sodium cyanoborohydride (7.2 g, 114.6 mmol). This product was carried forward without further purification. Structure and purity was confirmed by GC/MS. The retention time was 4.01 min. The MS was calculated for C9H11N 133, observed 133.

### b.) 2-methoxy-5-((3-methylindolin-1-yl)methyl)phenol

Following the same procedure as outlined in example 1, 3-methylindoline (1 g, 7.52 mmol) and isovanillin (1.15 g, 7.57 mmol) were dissolved in dichloromethane (37 ml) and treated with sodium triacetoxyborohydride (1.6 g, 7.57 mmol). Work up and chromatography provided the above titled product (1.24 g, 61% yield) as a tan solid.

¹H NMR (CDCl₃; 300 MHz) δ 1.27 (d, J=7 Hz, 3H), 2.79 (t, J=8 Hz, 1H), 3.21-3.30 (m, 1H), 3.47 (t, J=8 Hz, 1H), 3.83 (s, 3H), 3.99 (d, J=14 Hz, 1H), 4.24 (d, J=14 Hz, 1H), 6.47-6.50 (m, 1H), 6.64-6.70 (m, 1H), 6.75-6.84 (m, 2H), 6.93 (d, J=2 Hz, 1H), 7.01-7.18 (m, 2H); ¹³C NMR (CDCl₃; 75 MHz) δ 18.8, 35.3, 53.1, 56.1, 61.6, 107.2, 110.8, 114.5, 117.8, 119.6, 123.3, 127.5, 131.9, 135.2, 145.8, 145.9, 152.3; MS calculated for C17H19NO2+H 270, observed 270.

### EXAMPLE 6

### 2-methoxy-5-((5-methylindolin-1-yl)methyl)phenol

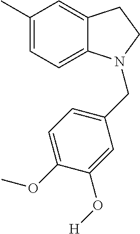

Following the same procedure as outlined in example 1, 5-methylindoline (2 g, 15 mmol) and isovanillin (2.28 g, 15 mmol) were dissolved in dichloromethane (75 ml) and treated with sodium triacetoxyborohydride (3.18 g, 15 mmol). Work up and chromatography provided the above titled product (2.08 g, 51% yield) as a light tan solid.

¹H NMR (CDCl₃; 300 MHz) δ 2.23 (s, 3H), 2.88 (t, J=8 Hz, 2H), 3.22 (t, J=8 Hz, 2H), 3.86 (s, 3H), 4.08 (s, 2H), 5.59 (br s, 1H), 6.41 (d. J=8 Hz, 1H), 6.76-6.85 (m, 3H), 6.90-6.94 (m, 2H); ¹³C NMR (CDCl₃; 75 MHz) δ 20.9, 28.7, 53.9, 54.1, 56.2, 107.3, 110.8, 114.5, 119.7, 125.6, 127.2, 127.6, 130.6, 132.1, 145.8. 145.9, 150.6; MS calculated for C17H19NO2+H 270, observed 270.

### EXAMPLE 7

### 2-methoxy-5((4-methylindolin-1-yl)methyl)phenol

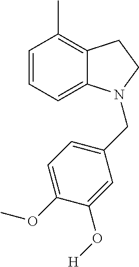

### a.) 4-methylindoline

Following the same procedure outlined in step a of example 3, 3-methylindole (5 g, 38.2 mmol) in glacial acetic acid (100 ml) was treated with sodium cyanoborohydride (7.2 g, 114.6 mmol). This product was carried forward without further purification. Structure and purity was confirmed by GC/MS. The retention time was 4.62 min. The MS was calculated for C9H11N 133, observed 133.

### b.) 2-methoxy-5-((4-methylindolin-1-yl)methyl)phenol

Following the same procedure as outlined in example 1, 4-methylindoline (1 g, 7.52 mmol) and isovanillin (1.14 g, 7.52 mmol) were dissolved in dichloromethane (38 ml) and treated with sodium triacetoxyborohydride (1.59 g, 7.52 mmol). Work up and chromatography provided the above titled product (1.26 g, 62% yield) as a white solid.

¹H NMR (CDCl₃; 300 MHz) δ 2.19 (s, 3H), 2.86 (t, J=8 Hz, 2H), 3.29 (t, J=8 Hz, 2H), 3.87 (s, 3H), 4.13 (s, 2H), 5.56 (s, 1H), 6.35 (d, J=8 Hz, 1H), 6.48 (d. J=8 Hz, 1H), 6.77-6.85 (m, 2H). 6.93-6.98 (m, 2H); ¹³C NMR (CDCl₃; 75 MHz) δ 18.7, 27.4, 53.3, 53.4. 56.2, 104.8. 110.8, 114.4, 119.2. 119.6, 127.6, 128.7, 132.1, 134.1, 145.8, 152.5; MS calculated for C17H19NO2+H 270, observed 270.

### EXAMPLE 8

### 2-methoxy-5-((6-methylindolin-1-yl)methyl)phenol

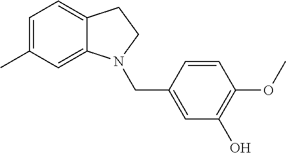

### a.) 6-methylindoline

Following the same procedure outlined in step a of example 3, 3-methylindole (4.5 g, 34.3 mmol) in glacial acetic acid (100 ml) was treated with sodium cyanoborohydride (6.5 g, 102.9 mmol). This product was carried forward without further purification. Structure and purity was confirmed by GC/MS. The retention time was 5.95 min. The MS was calculated for C9H11N 133, observed 133.

### b.) 2-methoxy-5-((6-methylindolin-1-yl)methyl)phenol

Following the same procedure as outlined in example 1, 6-methylindoline (1 g, 7.52 mmol) and isovanillin (1.14 g, 7.52 mmol) were dissolved in dichloromethane (38 ml) and treated with sodium triacetoxyborohydride (1.59 g, 7.52 mmol). Work up and chromatography provided the above titled product (1.68 g, 83% yield) as a light orange oil.

¹H NMR (CDCl₃; 300 MHz) δ 2.26 (s, 3H), 2.88 (t, J=8 Hz, 2H), 3.25 (t, J=8 Hz, 2H), 3.85 (s. 3H), 4.11 (s, 2H), 5.57 (br s, 1H), 6.34 (s, 1H), 6.47 (dd, J₁=7 Hz, J₂=1 Hz, 1H, 6.76-6.85 (m, 2H), 6.93-6.97 (m, 2H); ¹³C NMR (CDCl₃; 75 MHz) δ 21.9, 28.3, 53.3, 53.9, 56.2. 108.1, 110.8, 114.4, 118.4, 119.5, 124.3, 127.3, 132.0, 137.2, 145.8, 145.9, 152.9; MS calculated for C17H19NO2+H 270, observed 270.

### EXAMPLE 9

### 5-((6-fluoroindolin-1-yl)methyl)-2-methoxyphenol

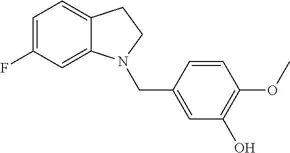

### a.) 6-fluoroindoline

Following the same procedure outlined in step a of example 3, 6-fluoroindole (5 g, 38.2 mmol) in glacial acetic acid (100 ml) was treated with sodium cyanoborohydride (7.2 g, 114.6 mmol). This product was carried forward without further purification. Structure and purity was confirmed by GC/MS. The retention time was 3.96 min. The MS was calculated for C9H11N 137, observed 137.

### b.) 5-((6-fluoroindolin-1-yl)methyl)-2-methoxyphenol

Following the same procedure as outlined in example 1, 6-fluoroindoline (1 g, 7.3 mmol) and isovanillin (1.12 g, 7.35 mmol) were dissolved in dichloromethane (37 ml) and treated with sodium triacetoxyborohydride (1.56 g, 7.35 mmol). Work up and chromatography provided the above titled product (1.08 g, 54% yield) as an amber oil.

¹H NMR (CDCl₃; 300 MHz) δ 2.88 (t, J=8 Hz, 2H), 3.34 (t, J=8 Hz, 2H), 3.85 (s, 3H), 4.10 (s, 2H), 5.66 (br s, 1H), 6.16 (dd, J₁=10 Hz, J₂=2 Hz, 1H), 6.27 (ddd, J₁=10 Hz, J₂=8 Hz, J₃=2 Hz, 1H), 6.79 (d, J=1 Hz, 2H), 6.90-6.94 (m, 2H): ¹³C NMR (CDCl₃; 75 MHz) δ 27.9, 52.7, 54.0, 56.2, 94.9, 95.2, 102.9, 103.2, 110.8, 114.3, 119.5, 124.6, 124.7, 125.29, 125.32, 131.2, 145.9, 146.0, 154.1, 154.2. 162.1, 165.2: MS calculated for C16H16FNO2+H 274, observed 274.

### EXAMPLE 10

### 2-methoxy-5-((6-methoxyindolin-1-yl)methyl)phenol

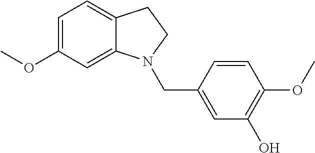

### a.) 6-methoxyindoline

Following the same procedure outlined in step a of example 3, 6-methoxyindole (5 g, 34 mmol) in glacial acetic acid (100 ml) was treated with sodium cyanoborohydride (6.43 g, 102 mmol). This product was carried forward without further purification.

### b.) 2-methoxy-5-((6-methoxyindolin-1-yl)methyl)phenol

Following the same procedure as outlined in example 1, 6-methoxyindoline (1 g, 6.7 mmol) and isovanillin (1.02 g, 6.70 mmol) were dissolved in dichloromethane (38 ml) and treated with sodium triacetoxyborohydride (1.4 g, 6.7 mmol). Work up and chromatography provided the above titled product (1.0 g, 52% yield) as a peach colored solid.

¹H NMR (CDCl₃; 300 MHz) δ 2.87 (t, J=8 Hz, 2H), 3.29 (t, J=8 Hz, 2H), 3.73 (s, 3H), 3.87 (s, 3H), 4.12 (s, 2H), 5.58 (br s, 1H), 6.09 (d, J=2 Hz, 1H), 6.16 (dd, J₁=8 Hz, J₂=2 Hz, 1H), 6.77-6.84 (m, 2H), 6.92-6.95 (m, 2H); ¹³C NMR (CDCl₃; 75 MHz) δ 27.9, 53.0, 54.2, 55.6, 56.2, 94.9, 101.6, 110.8, 114.4, 119.6, 122.6, 124.6, 131.8, 145.89, 145.92, 154.0, 160.3: MS calculated for C17H19NO3+H 286, observed 286.

### EXAMPLE 11

### Taste Evaluation

1% solutions of compounds of formula (I) were prepared in ethanol. These 1% solutions were then further diluted, using water, to create sample solutions of differing ppm concentrations.

A panel of trained experts was then asked to evaluate the taste of each sample solution and to comment on any inherent sweetness, other perceived taste, or flavor attributes.

After tasting each sample the experts rinsed their mouths with water and a break was observed until the stimulus disappeared.

The samples solutions were found to be sweet.

1. A compound of formula (I) 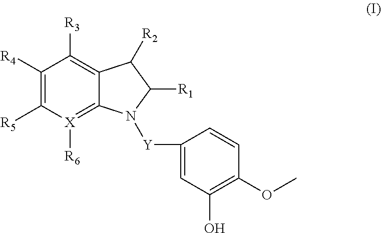
wherein:
X is N, or C,
R₁₋R₆ may be independently selected from the group consisting of H, substituted, unsubstituted, branched or unbranched C₁-C₄ alkyl, methoxy, ethoxy, Br, F, and Cl, provided that when X is nitrogen there is no substituent at R₆,
Y is an alkylene group of the formula (CR₇ R₈)ₘ wherein
R₇ and R₈ may be independently selected from the group consisting of hydrogen, and substituted, unsubstituted, branched or unbranched C₁-C₄ alkylene, and m is an integer selected from 1 to 6.

2. A compound according to claim 1 selected from the group consisting of: 5-(indolin-1-ylmethyl)-2-methoxyphenol, 2-methoxy-5-((7-methylindolin-1-yl)methyl)phenol, 2-methoxy-5-((4-methylindolin-1-yl)methyl)phenol, 2-methoxy-5-((6-methylindolin-1-yl)methyl)phenol, and 2-methoxy-5((5-methylinolin-1-yl)methyl)phenol.

3. A composition comprising a compound as defined in claim 1.

4. A composition comprising a compound as defined in claim 2.

5. A method for creating or modifying the flavour of a composition or consumable product comprising adding to said composition or consumable product at least one compound as defined in claim 1.

6. A method for creating or modifying the flavour of a composition or consumable product comprising adding to said composition or consumable product at least one compound as defined in claim 2.

7. A consumable product comprising a compound as defined in claim 1.

8. A consumable product comprising a compound as defined in claim 2.

9. A consumable product as defined in claim 7 comprising the compound at a concentration of 0.01 ppm-500 ppm based on the weight of the consumable product.

10. A consumable product as defined in claim 8 comprising the compound as defined in claim 2 at a concentration of 0.01 ppm-500 ppm based on the weight of the consumable product.

11. A method for creating or modifying flavour, optionally sweet flavour, of a consumable product comprising adding to said consumable product at least one compound as defined in claim 1.

12. A method for creating or modifying flavour, optionally sweet flavour, of a consumable product comprising adding to said consumable product at least one compound as defined in claim 2.

13. A consumable product as defined in claim 7 wherein the consumable product is selected from the group consisting of foodstuffs, confectionary products, baked products, sweet products, savoury products, dairy products, beverages and oral care products.

14. A consumable product as defined in claim 8 wherein the consumable product is selected from the group consisting of foodstuffs, confectionary products, baked products, sweet products, savoury products, dairy products, beverages and oral care products.
